(19) United States
(12) Patent Application Publication
(10) Pub. No.: US 2024/0122132 A1
(43) Pub. Date: Apr. 18, 2024

(54) Enriched Coir

(71) Applicant: Fibredust, LLC, Cromwell, CT (US)

(72) Inventors: Pazhanimuthu Annamalai, Chennai (IN); Satheesh Rao, Pollachi (IN)

(73) Assignee: Fibredust, LLC, Cromwell, CT (US)

(21) Appl. No.: 18/372,358

(22) Filed: Sep. 25, 2023

Related U.S. Application Data
(60) Provisional application No. 63/409,249, filed on Sep. 23, 2022.

(51) Int. Cl. A01G 24/25; A01G 24/15; A01G 24/42

(52) U.S. Cl. CPC A01G 24/25; A01G 24/15; A01G 24/42

(57) ABSTRACT

The present subject matter is directed to a growing medium and method of use. The growing medium comprises coconut coir. The coconut coir has a specific range of electrolytes, particularly sodium, potassium, and calcium. The values of these elements is confirmed in the coconut coir through qualitative and quantitative measurements, such as photoelectric flame photometry, spectroscopy, or inductively coupled plasma atomic emission spectroscopy.

### PRIORITY

This application claims priority to U.S. Provisional Patent Application Ser. No. 63/409,249 filed on Sep. 23, 2022, the disclosure of which is hereby fully incorporated by reference.

### FIELD OF THE INVENTION

The present subject matter pertains generally to a coconut coir product and a method of making a coconut coir product. More specifically, the present subject matter is directed to a coconut coir product usable as a plant growing medium or as a soil amendment.

### BACKGROUND OF INVENTION

Coir is a natural fiber extracted from the outer husk of coconut. Coconut coir can be processed into a coconut coir product usable as a plant growing medium or as a soil amendment.

The amount and kind of processing needed to render the raw coconut coir into a product usable as a plant growing medium or as a soil amendment, and thereby the cost to so process, depends greatly on the sodium and potassium content of the raw coconut coir. High potassium and sodium content can render the coconut coir product as undesirable as a plant growing medium or as a soil amendment for a variety of reasons. For example, too much potassium can interfere with magnesium availability. At the same time, it is desirable to be able to source coconut coir with high potassium and sodium content because of these problems because it is in lower demand and therefore less expensive than other raw coconut coir having lower potassium and sodium content.

It remains desirable to develop an inexpensive and high quality coconut coir product usable as a plant growing medium or as a soil amendment with acceptably low potassium and sodium content. It is especially desirable to develop processes by which raw coconut coir with high potassium or sodium content can be processed into an inexpensive and high quality coconut coir product usable as a plant growing medium or as a soil amendment with acceptably low potassium and sodium content.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The present subject matter is directed to a coconut coir product comprising coconut coir having less than 100 ppm sodium as measured by photoelectric flame photometry, or flame emission spectroscopy, or inductively coupled plasma atomic emission spectroscopy (ICP-AES).

The present subject matter is also directed to a coconut coir product comprising coconut coir having less than 350 ppm potassium as measured by photoelectric flame photometry, or flame emission spectroscopy, or inductively coupled plasma atomic emission spectroscopy.

The present subject matter is also directed to a coconut coir product comprising coconut coir having greater than 850 ppm calcium as measured by photoelectric flame photometry, or flame emission spectroscopy, or inductively coupled plasma atomic emission spectroscopy.

The invention is directed toward a growing medium comprising coconut coir wherein said coconut coir comprises less than 100 ppm sodium; wherein said coconut coir comprises less than 350 ppm potassium; and wherein said coconut coir comprises less than 850 ppm calcium.

In other embodiments the coconut coir has an average particle size between 4.0 mm and 5.0 mm. In other embodiments the coconut coir comprises at least 50 ppm sodium; at least 175 ppm potassium; and at least 425 ppm calcium.

The amounts of sodium, potassium, and calcium may be confirmed through measurement by spectroscopy, photoelectric flame photometry, flame emission spectroscopy, or inductively coupled plasma atomic emission spectroscopy.

The invention is also directed toward a method for enhancing plant growth comprising providing a growing medium comprising coconut coir, wherein said coconut coir comprises less than 100 ppm sodium; wherein said coconut coir comprises less than 350 ppm potassium; and wherein said coconut coir comprises less than 850 ppm calcium. The method may further comprise placing a plant seedling within the growing medium.

The invention is also directed toward a method for creating a growing medium comprising obtaining coconut coir; creating a treatment solution by Combining water with 16% v/v calcium metasilicate and phosphoric acid; combining said coconut coir with said treatment solution; waiting a predetermined period of time to permit saturation of said coconut coir by said treatment solution; testing a sample of said coconut coir after said predetermined period of time; confirming that said sample of said coconut coir comprises less than 100 ppm of sodium; confirming that said sample of said coconut coir comprises less than 350 ppm of potassium; and confirming that said sample of said coconut coir comprises less than 850 ppm of calcium.

Still other embodiments of the present invention will become readily apparent to those skilled in this art from the following description wherein there is shown and described the embodiments of this invention, simply by way of illustration of the best modes suited to carry out the invention. As it will be realized, the invention is capable of other different embodiments and its several details are capable of modifications in various obvious aspects all without departing from the scope of the invention. Accordingly, the drawing and descriptions will be regarded as illustrative in nature and not as restrictive.

### BRIEF DESCRIPTION OF THE DRAWINGS

Various exemplary embodiments of this invention will be described in detail, wherein like reference numerals refer to identical or similar components, with reference to the following figures, wherein:

FIG. 1 is a flow diagram of a first embodiment of a process for production of coconut coir;

FIG. 2 is a block diagram of husks and debris on a surface;

FIG. 3 is a diagram of a sieving step;

FIG. 4 is a diagram of a drying step;

FIG. 5 is a diagram of a treatment mixture;

FIG. 6 is a diagram of a combination in a bunker; and

FIG. 7. is a flowchart of a rinsing process.

### DETAILED DESCRIPTION OF THE PREFERRED EMBODIMENT

The claimed subject matter is now described with reference to the drawings. In the following description, for purposes of explanation, numerous specific details are set forth in order to provide a thorough understanding of the claimed subject matter. It may be evident, however, that the claimed subject matter may be practiced with or without any combination of these specific details, without departing from the spirit and scope of this invention and the claims.

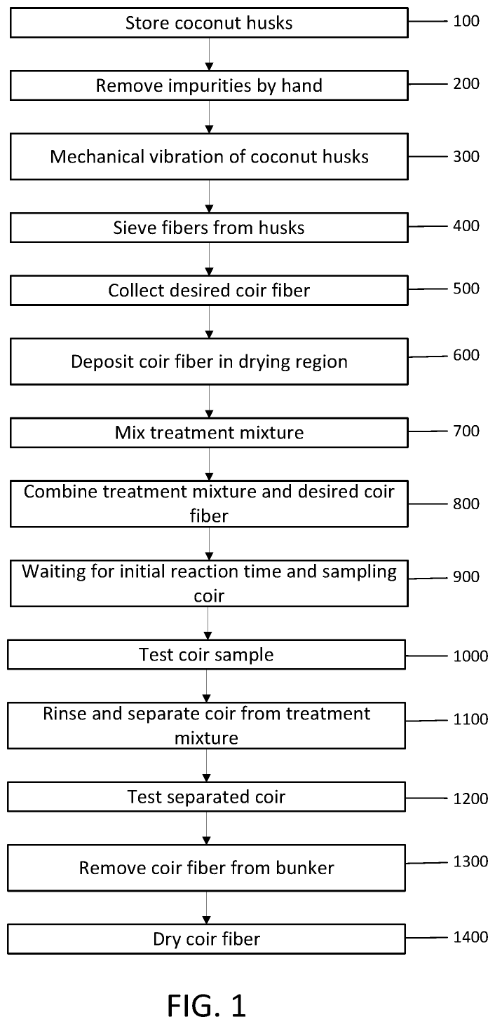

Referring to FIG. 1 shown is a flow diagram of a first embodiment of a process for production of a high quality coconut coir product. It should be understood that the first embodiment of a process may comprise 14 steps, which will be detailed herebelow, but that the first embodiment of a process may include additional steps chosen with good engineering judgment. Further in some optional embodiments, one or more of the 14 steps may be optional and omitted or modified.

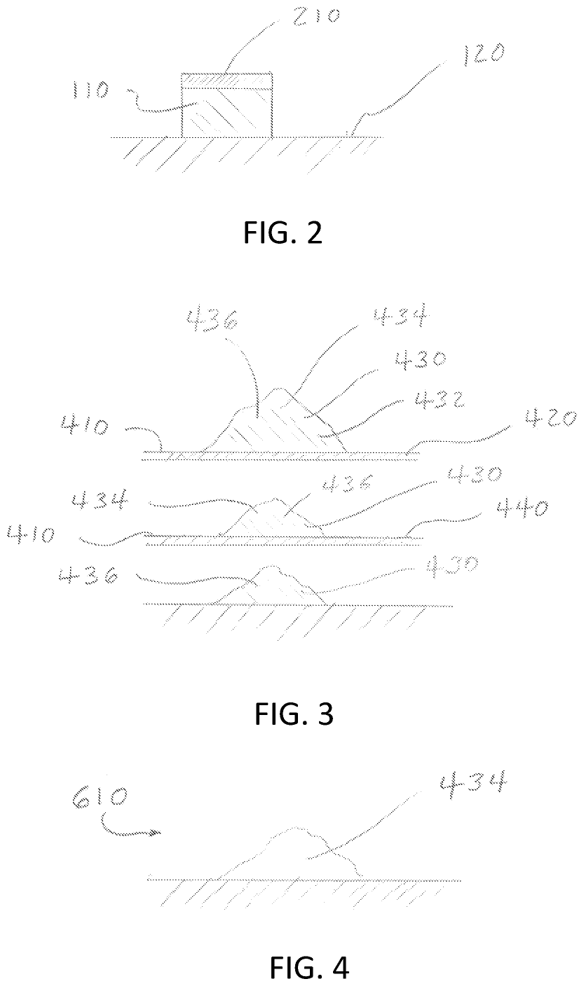

With continued reference to FIG. 1, and with reference to FIG. 2, the first step 100 may comprise storing coconut husks 110 on a concrete surface or on weed or straw mats or other reasonably clean surface 120. Step 100 is taken, in part, to prevent or reduce soil contamination of the husks. In some circumstances, step 100 is optional and may be omitted. For example, where the husks are to be processed immediately such that soil contamination is unlikely, step 100 may be unnecessary.

With continued reference to FIGS. 1 and 2, the second step 200 and third step 300 may comprise separation and removal of impurities 210 from the coconut husks 110. The steps for separation of impurities 210 from the husk may involve removal of undesired soil, mud, insects, leaves, or other debris. Step 200 may comprise removal of impurities by hand. Step 300 may comprise shaking of the husks, vibration of the husks, beating of the husks, or a combination thereof.

With continued reference to FIG. 1, the fourth step 400 may comprise sieving fibers 430 from the coconut husks 110 to yield desired size coir fiber 434. Sieving fibers 430 from the coir may be done with a sieve 410 such as, and without limitation, a stationary sieve, rotating sieve, a rotating sieve with an auger or set of auger plates, a vibrating sifter, or similar processing where the coir is moved over a mesh which permits or induces smaller materials (e.g. fibers) to pass while retaining the larger materials (e.g. fibers). In certain embodiments the material may be sieved at least twice: a first time with a first sieve 420 to remove large fibers 432 above a maximum desired size and a second time with a second sieve 440 to remove small fibers 436 below a minimum desired size. With reference now to FIG. 3, shown is a first sieve 420 upon which fibers 430 are deposited and through which the desired size coir fiber 434 and small fibers 436 below the minimum desired size can pass, and which will retain the large fibers 432 above the maximum desired size. Beneath the first sieve 420 is a second sieve 440 upon which the fibers 430 passing the first sieve 420 are deposited and through which the small fibers 436 below the minimum desired size can pass and which will retain the desired size coir fiber 434. Using these latter sieves, the desired size coir fiber 434 may be separated from the large fibers 432 and the small fibers 436.

With continued reference to FIG. 1, the fifth step 500 may comprise collecting the desired size coir fiber 434 and transporting it. Collection and transport of the desired size coir fiber may be by conveyor or auger or hand or mechanical loader or truck or other means chosen with good engineering judgment.

With continued reference to FIG. 1 and with additional reference to FIG. 4, the sixth step 600 may comprise depositing the desired size coir fiber 434 in a drying region 610. The drying region may be a dryer or drying floor or drying yard or other region chosen with good engineering judgment to remove water from the desired size coir fiber 434. Without limitation, the drying region 610 may be a closed region with dry air circulated therein, or a closed region with warm and dry air circulated therein, or a drying yard wherein drying action is provided by sunlight and open air. Further to the sixth step, in certain embodiments, the desired size coir fiber 434 remains in the drying region 610 until a desired dryness of the desired size coir fiber 434 results. In certain embodiments, and without limitation, the desired size coir fiber 434 in the drying region is periodically tested for water content. In other embodiments, the humidity of the drying region is measured and the residence time of the coir therein is calculated to produce a desired dryness of the coir. In some embodiments, and without limitation, the desired size coir fiber 434 may be periodically or continuously agitated during drying. If the desired size coir fiber is already of sufficient dryness after the step(s) proceeding step 600, then step 600 may optionally be omitted.

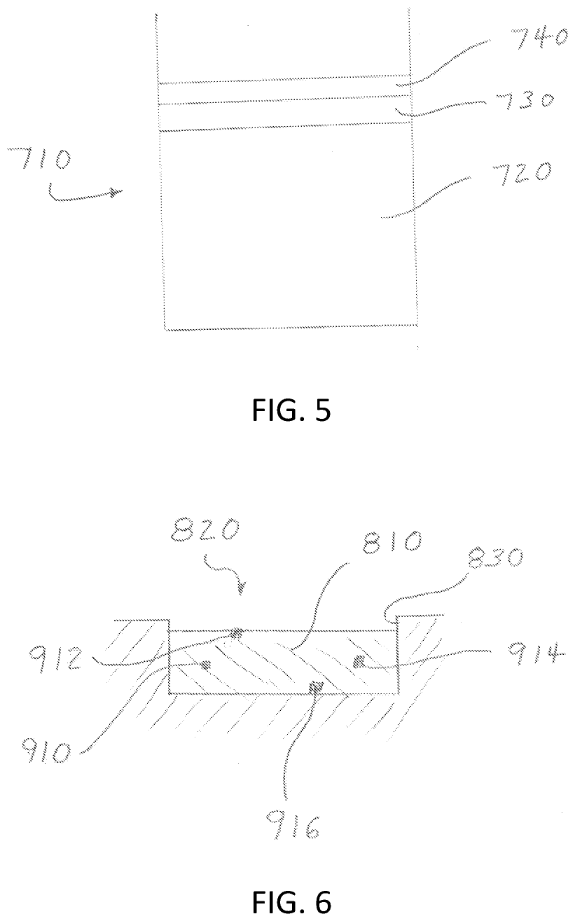

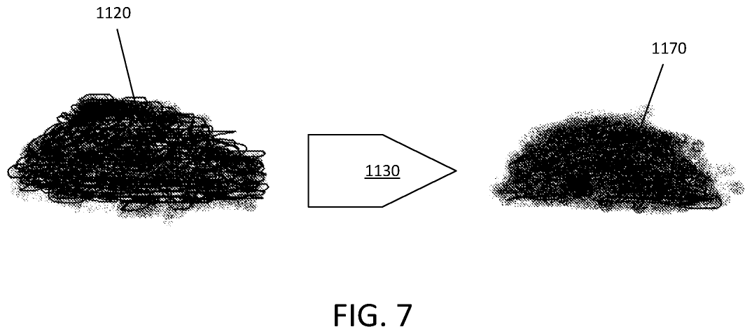

With continued reference to FIG. 1, and with additional reference to FIG. 5, the seventh step 700 may comprise mixing a treatment mixture 710. A treatment mixture 710 may be prepared by combining water 720 with a calcium metasilicate solution in water 730 and phosphoric acid 740 and thoroughly mixing the combination. In some embodiments, a treatment mixture 710 may be prepared by combining 45,000 liters of water 720 with 700 kilograms of a 16% v/v calcium metasilicate solution in water 730 and 20 liters of phosphoric acid 740 and thoroughly mixing the combination. In some embodiments some or all of the water 720 used in forming the treatment mixture 710 may be reverse osmosis water, or distilled water, or deionized water. Other acceptable treatment mixtures may be prepared by combining more or less of the latter components in substantially the same ratio by volume. As used herein and unless otherwise noted, substantially the same ratio is any ratio wherein the amounts are within 5% of each of the recited amounts. In some embodiments, the treatment mixture 710 is agitated for 5 minutes after being thoroughly mixed. In some embodiments, the treatment mixture 710 is agitated for 10 minutes after being thoroughly mixed. In some embodiments, the treatment mixture 710 is agitated for 20 minutes after being thoroughly mixed. In some embodiments, the treatment mixture 710 is agitated for 30 minutes after being thoroughly mixed. It should be understood that the treatment mixture 710 does not have to be prepared after the prior listed steps 100-600. To the contrary, the treatment mixture 710 may be prepared before, during, or after any of steps 100-600.

With continued reference to FIG. 1, and with additional reference to FIG. 6, the eighth step 800 may comprise mixing the treatment mixture 710 with the desired size coir fiber 434. In some embodiments, and without limitation, the combination 810 of treatment mixture 710 and the desired size coir fiber 434 may be mixed in a bunker 820. The bunker 820 may be any sufficiently secure structure that will retain the deposited combination 810 during subsequent treatment while excluding undesirable debris or other material being introduced. In one non-limiting embodiment, a bunker 820 may comprise a concrete enclosure having a concrete floor and concrete walls adapted to retain the combination 810 during a treatment period. It should be understood that perfect retention of the combination 810 is not required and that some minor leakage or loss may be acceptable. The bunker 820 may optionally have an open top. In embodiments with bunker walls 830, the bunker walls 830 may be low enough to permit the desired size coir fiber to be deposited in the bunker 820 by hand or by conventional commercial earth moving equipment such as, and without limitation, a backhoe. The bunker 820 may either be open to the sun and weather or may be housed or roofed or sheltered from sun and weather. In some embodiments and without limitation, the bunker may be 2100 cubic feet. In some embodiments and without limitation, the bunker may be 2800 cubic feet. In some embodiments and without limitation, the bunker may be 3500 cubic feet. In some embodiments and without limitation, the bunker may be 4200 cubic feet. In some embodiments and without limitation, the bunker may be 4900 cubic feet or larger. In some embodiments, and without limitation the combination 810 is agitated to promote mixture of the treatment mixture 710 with the desired size coir fiber 434. The agitation may be periodic or continuous. The rate of agitation may be chosen with good engineering judgment. In some embodiments and without limitation, the combination 810 is a semi-soup texture when agitation is completed.

With continued reference to FIGS. 1 and 6, the ninth step 900 may comprise allowing the combination 810 to react for some initial treatment time. In some embodiments and without limitation, the initial treatment time is 2 hours. In some embodiments and without limitation, the initial treatment time is 4 hours. In some embodiments and without limitation, the initial treatment time is 6 hours. In some embodiments and without limitation, the initial treatment time is 8 hours. In some embodiments and without limitation, the initial treatment time is 10 hours. In some embodiments and without limitation, the initial treatment time is 12 hours or longer. The ninth step 900 may further comprise, after the initial treatment time, sampling. In one non-limiting embodiment of sampling, a sample 910 is drawn from some depth of the combination 810 and then washed thoroughly in distilled water to yield a washed coir sample. In an alternative non-limiting embodiment of sampling, multiple samples 910 are drawn from differing depths of the mixture of the treatment mixture and the coir and then each sample is washed thoroughly in distilled water to yield a washed coir sample. In one non-limiting embodiment of sampling a first sample 912 is drawn at the surface of the mixture, one or more other samples 914 are drawn at one or more intermediate depths between the surface of the mixture and the bottom of the mixture, and a final sample 916 is drawn from the bottom of the mixture.

With continued reference to FIG. 1, the tenth step 1000 may comprise testing one or more washed coir samples such as, and without limitation, one or more samples taken in the ninth step 900. In one embodiment testing includes washing a sample thoroughly in lab grade distilled water and then testing the washed sample by measuring for sodium value, potassium value, magnesium value, calcium value, or some combination of these values. Measuring for sodium value, potassium value, magnesium value, calcium value, or some combination of these values may be done by photoelectric flame photometry, or flame emission spectroscopy, or inductively coupled plasma atomic emission spectroscopy (ICP-AES), or other means chosen with good engineering judgment. The result of the measurement for sodium value will be some number of parts per million (ppm). The result of the measurement for potassium value will be some number of parts per million (ppm). The result of the measurement for magnesium value will be some number of parts per million (ppm). The result of the measurement for calcium value will be some number of parts per million (ppm).

In some embodiments one or more of the above measured values, sodium value, potassium value, magnesium value, calcium value, are compared to one or more standards to determine if the mixture of the treatment mixture and the coir has had sufficient time to react. If all of the standards are met, the combination 810 has had sufficient time to react, the reaction is complete and the process may be advanced to one or more subsequent post-reaction steps. If one or more of the standards are not met, then combination 810 has not had sufficient time to react and the combination 810 may be given more time to react. In some embodiments, where the combination 810 is given more time to react, additional sampling is done every hour, or at some other interval chosen with good engineering judgment to determine if the reaction is complete. In some non-limiting embodiments, a standard is directed to the sodium value. In some embodiments with a standard directed to a sodium value, the standard is that the sodium value be less than 200 ppm. In some embodiments with a standard directed to a sodium value, the standard is that the sodium value be less than 100 ppm. In some embodiments with a standard directed to a sodium value, the standard is that the sodium value be less than 50 ppm. In some embodiments with a standard directed to a sodium value, the standard is that the sodium value be less than 25 ppm. In some non-limiting embodiments, a standard is directed to the potassium value. In some embodiments with a standard directed to a potassium value, the standard is that the potassium value be less than 450 ppm. In some embodiments with a standard directed to a potassium value, the standard is that the potassium value be less than 350 ppm. In some embodiments with a standard directed to a potassium value, the standard is that the potassium value be less than 250 ppm. In some embodiments with a standard directed to a potassium value, the standard is that the potassium value be less than 150 ppm. In some embodiments with a standard directed to a potassium value, the standard is that the potassium value be less than 50 ppm. In some non-limiting embodiments, a standard is directed to the magnesium value. In some embodiments with a standard directed to a magnesium value, the standard is that the magnesium value be less than 300 ppm. In some embodiments with a standard directed to a magnesium value, the standard is that the magnesium value be less than 250 ppm. In some embodiments with a standard directed to a magnesium value, the standard is that the magnesium value be less than 200 ppm. In some embodiments with a standard directed to a magnesium value, the standard is that the magnesium value be less than 150 ppm. In some non-limiting embodiments, a standard is directed to the calcium value. In some embodiments with a standard directed to a calcium value, the standard is that the calcium value be less than 1150 ppm. In some embodiments with a standard directed to a calcium value, the standard is that the calcium value be less than 850 ppm. In some embodiments with a standard directed to a calcium value, the standard is that the calcium value be less than 550 ppm. In some embodiments with a standard directed to a calcium value, the standard is that the calcium value be less than 250 ppm.

In some embodiments, there are a plurality of standards using a standard directed to a sodium value, a standard directed to a potassium value, a standard directed to a magnesium value, a standard directed to a calcium value, or some combination thereof.

With continued reference to FIG. 1, once the reaction in the tenth step 1000 is complete, the process may move on to one or more of the post-reaction steps. The post-reaction steps may include one or more of the eleventh step 1100, the twelfth step 1200, the thirteenth step 1300, and the fourteenth step 1400.

With continued reference to FIG. 1, the eleventh step 1100 may comprise washing or rinsing the combination 810 to separate the now treated coir fiber 1120 from the treatment mixture 710. In some embodiments, the treated coir fiber 1120 is scooped out of the bunker 820 and rinsed in water, such as reverse osmosis water. In other embodiments the treatment mixture 710 is pumped out or siphoned out of the bunker 820 and the now treated coir fiber 1120 is rinsed in the bunker to remove residual treatment mixture 710. In some embodiments the used treatment mixture 710 may be recovered and partially or wholly reused or recycled. After undergoing a rinsing process 1130, the treated coir fiber 1120, is rinsed and treated coir fiber 1170.

With continued reference to FIG. 1, the twelfth step 1200 may comprise a testing one or more samples of the rinsed and treated coir fiber 1170 for a standard directed to a sodium value, a standard directed to a potassium value, a standard directed to a magnesium value, a standard directed to a calcium value, or some combination thereof using photoelectric flame photometry, or flame emission spectroscopy, or inductively coupled plasma atomic emission spectroscopy (ICP-AES), or other means chosen with good engineering judgment.

With continued reference to FIG. 1, the thirteen step 1300 may comprise removing the rinsed and treated coir fiber 1170 from the bunker 820. Removal of the rinsed and treated coir fiber 1170 may be done by hand or by hand tools or by equipment such as conventional commercial earth moving equipment such as, and without limitation, a backhoe.

With continued reference to FIG. 1, the fourteenth step 1400 may comprise drying the rinsed and treated coir fiber 1170. The drying in the fourteen step 1400 may be performed similarly to the drying in the sixth step 600. The rinsed and treated coir fiber 1170 may be placed in a drying region which may be a dryer or drying floor or drying yard or other region chosen with good engineering judgment to remove water from the rinsed and treated coir fiber 1170. The drying region may be a closed region with dry air circulated therein, or a closed region with warm and dry air circulated therein, or a drying yard wherein drying action is provided by sunlight and open air. In certain embodiments, the rinsed and treated coir fiber 1170 remains in the drying region until a desired dryness of the rinsed and treated coir fiber 1170 results. In certain embodiments, and without limitation, the rinsed and treated coir fiber 1170 in the drying region is periodically tested for water content. In other embodiments, the humidity of the drying region is measured and the residence time of the rinsed and treated coir fiber 1170 therein is calculated to produce a desired dryness of the rinsed and treated coir fiber 1170. In some embodiments, and without limitation, the rinsed and treated coir fiber 1170 may be periodically or continuously agitated during drying. If the rinsed and treated coir fiber 1170 is already of sufficient dryness after the step(s) proceeding step 1400, then step 1400 may optionally be omitted.

In short, the process for making the invention is completed by the following steps
1. Combining 45,000 liters of water with 700 kilograms of 16% v/v calcium metasilicate solution and 20 liters of phosphoric acid.
2. Mixing and agitating the solution thoroughly for up to 30 minutes.
3. Pouring the mixture over 4,200 cubic feet of coconut coir.
4. Allowing 8 hours for saturation and reaction of the mixture with the coconut coir.
5. Draw sample of coconut coir and wash with distilled water.
6. Test sample of coconut coir to ensure that coconut coir contains less than 100 ppm of sodium, less than 350 ppm of potassium, and less than 850 ppm of calcium.
7. If levels are too high then draining solution off of coconut coir and washing with water to lower the levels until the desired amounts are reached. The water utilized is preferably distilled via reverse osmosis prior to combination with the coconut coir. The water is preferably replaced in the solution in 1:1 ratio with less than 50 percent of the solution being removed at any one time. For instance, if 50 liters of solution are removed then 50 liters of water are added.
8. Draining solution from coconut coir and drying the coconut coir.

The final treated coconut coir preferably contains less than 100 ppm of sodium, less than 350 ppm of potassium, and less than 850 ppm of calcium in the preferred embodiment. In other embodiments there may be any amount of sodium greater than zero, any amount of potassium greater than zero, and/or any amount of calcium greater than zero. These amounts are greater than any amount naturally occurring in coconut coir.

The amounts of added sodium, potassium, and calcium are preferably measured by photoelectric flame photometry, flame emission spectroscopy, or inductively coupled plasma atomic emission spectroscopy (ICP-AES). Other methods may be utilized to test the amounts of added sodium, potassium, and calcium. One having skill in the art would understand that any quantitative analysis or qualitative analysis test may be used. One may use gravimetry, optical atomic spectroscopy, neutron activation analysis, mass spectrometric atomic spectroscopy, x-ray fluorescence, particle-induced x-ray emission, x-ray photoelectron spectroscopy, Auger electron spectroscopy, sodium fusion testing, Schöniger oxidation, electrochemical analysis, or any other known method of elemental analysis.

Other methods that may be utilized include, but are not limited to, utilizing a mass spectrometer, atomic absorption spectrometer, atomic and optical emission spectrometer, benchtop nuclear magnetic resolution (NMR) spectrometer, electron paramagnetic resonance (EPR) spectrometer, elemental analyzer, fluorescence spectrometer, FTIR spectrometer, FT-NIR Fourier transform spectrometer, gas chromatographer, gel permeation chromatographer, hyperspectral imager, ion chromatographer, liquid chromatographer, pharmaceutical analysis equipment, raman spectrometer, secondary ion mass spectrometer, vapor pressure measurement device, x-ray and gamma ray detector, x-ray diffractometer, x-ray fluorescence analyzer, x-ray photoelectron spectrometer, or XRF spectrometer.

The final coconut coir product may come in any form and have any particle size. In the preferred embodiment the final coconut coir has an average particle size of 0.5 mm to 1.0 mm. In another embodiment, the average particle size is 1.0 mm to 4.0 mm. In another embodiment the average particle size is 4.0 mm to 5.0 mm. In other embodiments the coconut coir has an average particle size of 0.1 mm to 0.2 mm. In other embodiments the coconut coir has an average particle size of 0.2 mm to 0.5 mm. In addition, the coconut coir may contain fibers of coconut coir. The coconut coir fibers may be any length. In the preferred embodiment the fibers are less than 2.0 cm long. In the preferred embodiment the coconut fibers make up no more than 13% by weight of the total amount of coconut coir.

A person may use the treated coconut coir in any manner. In the preferred manner the user obtains a brick of the treated coconut coir. The user breaks up the brick into smaller pieces to create a loose grouping of the treated coconut coir. The user places the treated coconut coir into a growing pot or in a hole in the ground. The user may also combine the treated coconut coir with natural soil or additional fertilizer. The user may then place a seed or a seedling into the treated coconut coir. The user may apply a desired amount of water to the treated coconut coir to a desired saturation level.

What has been described above includes examples of the claimed subject matter. It is, of course, not possible to describe every conceivable combination of components or methodologies for purposes of describing the claimed subject matter, but one of ordinary skill in the art can recognize that many further combinations and permutations of such matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

The foregoing method descriptions and the process flow diagrams are provided merely as illustrative examples and are not intended to require or imply that the steps of the various embodiments must be performed in the order presented. As will be appreciated by one of skill in the art the order of steps in the foregoing embodiments may be performed in any order. Words such as “thereafter,” “then,” “next,” etc. are not intended to limit the order of the steps; these words are simply used to guide the reader through the description of the methods. Further, any reference to claim elements in the singular, for example, using the articles “a,” “an” or “the” is not to be construed as limiting the element to the singular.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

1. A growing medium comprising
a) coconut coir
b) wherein said coconut coir comprises less than 100 ppm sodium;
c) wherein said coconut coir comprises less than 350 ppm potassium; and
d) wherein said coconut coir comprises less than 850 ppm calcium.

2. The growing medium as in claim 1 wherein said coconut coir has an average particle size between 4.0 mm and 5.0 mm.

3. The growing medium as in claim 1
a) wherein said coconut coir comprises at least 50 ppm sodium;
b) wherein said coconut coir comprises at least 175 ppm potassium; and
c) wherein said coconut coir comprises at least 425 ppm calcium.

4. The growing medium as in claim 1 wherein the amounts of sodium, potassium, and calcium are confirmed through measurement by spectroscopy.

5. The growing medium as in claim 1 wherein the amounts of sodium, potassium, and calcium are confirmed through measurement by photoelectric flame photometry.

6. The growing medium as in claim 1 wherein the amounts of sodium, potassium, and calcium are confirmed through measurement by flame emission spectroscopy.

7. The growing medium as in claim 1 wherein the amounts of sodium, potassium, and calcium are confirmed through measurement by inductively coupled plasma atomic emission spectroscopy.

8. A method for enhancing plant growth comprising:
a) providing a growing medium,
b) wherein said growing medium comprises coconut coir;
c) wherein said coconut coir comprises less than 100 ppm sodium;
d) wherein said coconut coir comprises less than 350 ppm potassium; and
e) wherein said coconut coir comprises less than 850 ppm calcium.

9. The method as in claim 8 further comprising placing a plant seedling within the growing medium.

10. A method for creating a growing medium comprising
a) obtaining coconut coir;
b) creating a treatment solution by Combining water with 16% v/v calcium metasilicate and phosphoric acid;
c) combining said coconut coir with said treatment solution;
d) waiting a predetermined period of time to permit saturation of said coconut coir by said treatment solution;
e) testing a sample of said coconut coir after said predetermined period of time;
f) confirming that said sample of said coconut coir comprises less than 100 ppm of sodium;
g) confirming that said sample of said coconut coir comprises less than 350 ppm of potassium; and
h) confirming that said sample of said coconut coir comprises less than 850 ppm of calcium.

11. The method as in claim 10 wherein the step of testing comprises testing by spectroscopy.

12. The method as in claim 10 wherein the step of testing comprises testing by photoelectric flame photometry.

13. The method as in claim 10 wherein the step of testing comprises testing by flame emission spectroscopy.

14. The method as in claim 10 wherein the step of testing comprises testing by inductively coupled plasma atomic emission spectroscopy.
